(19) United States
(12) Patent Application Publication
(10) Pub. No.: US 2025/0120898 A1
(43) Pub. Date: Apr. 17, 2025

(54) COSMETIC COMPOSITION, COSMETIC, METHOD FOR APPLYING COSMETIC COMPOSITION, AND METHOD FOR APPLYING COSMETIC

(71) Applicant: UNDER7 Co., Ltd., Tokyo (JP)

(72) Inventor: Keita MINAMI, Tokyo (JP)

(21) Appl. No.: 18/786,850

(22) Filed: Jul. 29, 2024

(30) Foreign Application Priority Data
Oct. 17, 2023 (JP) 2023-178555

(51) Int. Cl. A61K 8/365; A61K 8/23; A61K 8/44; A61Q 5/00; A61Q 19/00

(52) U.S. Cl. CPC A61K 8/365; A61K 8/23; A61K 8/44; A61Q 5/00; A61Q 19/00

(57) ABSTRACT

A cosmetic composition comprises tartaric acid, glycine, Na citrate, alanine, 2Na succinate, and Na sulfate, wherein an amount of the tartaric acid is 57.07% by mass based on the total mass of the composition, an amount of the glycine is 13.42% by mass based on the total mass of the composition, an amount of the Na citrate is 10.41% by mass based on the total mass of the composition, an amount of the alanine is 7.61% by mass based on the total mass of the composition, an amount of the 2Na succinate is 7.48% by mass based on the total mass of the composition, and an amount of the Na sulfate is 4.01% by mass based on the total mass of the composition.

### CROSS-REFERENCE TO RELATED APPLICATION

This application is based on and claims priority to Japanese Patent Application No. 2023-178555 filed on Oct. 17, 2023, the entire disclosures of which are hereby incorporated by reference herein.

### BACKGROUND OF THE INVENTION

### Field of the Invention

The present invention relates to a cosmetic composition, a cosmetic comprising this cosmetic composition, and a method for applying this cosmetic composition or cosmetic.

### Description of the Related Art

Cosmetic compositions supplemented with various substances have heretofore been known for the purpose of imparting specific effects to cosmetics. For example, cosmetic compositions supplemented with nucleic acid or citric acid are known in the art in order to improve the texture of hair of the head or the scalp.

For example, there exists Japanese Patent Laid-Open No. 2018-203628 as a prior art document that discloses this kind of cosmetic composition. The invention disclosed in Japanese Patent Laid-Open No. 2018-203628 can reportedly provide a cosmetic composition or a food or drink composition containing a substance that provides resilience or gloss to the skin or hair of the head and offers very favorable touch or usability of the skin or hair of the head, such as an improved moisturizing effect, whitening effect, or combability of hair, when the composition is used externally or internally.

However, conventional techniques typified by Japanese Patent Laid-Open No. 2018-203628 described above, in many cases, cannot sufficiently exert their effects even if cosmetics are supplemented with components such as nucleic acid or citric acid. The conventional techniques have found a solution by an approach such as increase in the amount of nucleic acid, citric acid, or the like added and however, present problems associated with cost and stability in formulations. Particularly, for improvement in the texture of hair of the head or the scalp, it is necessary to optimize substance components constituting a cosmetic composition. Thus, there has been a demand for the achievement of a novel cosmetic composition that exerts a high improving effect.

The present applicant has then proposed the invention described in Japanese Patent Laid-Open No. 2023-107183, the invention relating to a cosmetic composition for solving the problems with the conventional techniques. The invention described in Japanese Patent Laid-Open No. 2023-107183 comprises nucleic acid as a main constituent component. However, cosmetic compositions and cosmetics comprising nucleic acid in constituent components, while satisfy the domestic laws and standards of Japan, have not been sometimes approved for use in foreign countries, for example, the United States of America. The present inventor has decided to again invent a cosmetic composition or a cosmetic that comprises no nucleic acid and that can be carried out in foreign countries, for example, the United States of America. It has been necessary for such an invention that not only nucleic acid be removed from the invention described in Japanese Patent Laid-Open No. 2023-107183, but also the fundamental design of components be reviewed.

The present invention has been made in light of the problems with the conventional techniques mentioned above. An object of the present invention is to provide a cosmetic composition having a novel formulation of components that can exert a high improving effect on the texture of hair of the head or the scalp, a cosmetic comprising this cosmetic composition, and a method for applying this cosmetic composition or cosmetic. Another object of the present invention is to provide a cosmetic composition that meets the laws and standards of foreign countries other than Japan and thus is usable in the world, a cosmetic comprising this cosmetic composition, and a method for applying this cosmetic composition or cosmetic.

### SUMMARY OF THE INVENTION

The cosmetic composition according to the present invention is a cosmetic composition comprising
tartaric acid,
glycine,
Na citrate,
alanine,
2Na succinate, and
Na sulfate, wherein
an amount of the tartaric acid is 57.07% by mass based on the total mass of the composition,
an amount of the glycine is 13.42% by mass based on the total mass of the composition,
an amount of the Na citrate is 10.41% by mass based on the total mass of the composition,
an amount of the alanine is 7.61% by mass based on the total mass of the composition,
an amount of the 2Na succinate is 7.48% by mass based on the total mass of the composition, and
an amount of the Na sulfate is 4.01% by mass based on the total mass of the composition.

The cosmetic according to the present invention can be formed by incorporating one or more members selected from histidine, proline, isoleucine, serine, valine, aspartic acid, threonine, phenylalanine, arginine, PCA, PCA-Na, Na lactate, and water in the cosmetic composition according to claim 1.

The cosmetic according to the present invention can be configured such that
an amount of the tartaric acid is 57% by mass based on the total mass of the cosmetic,
an amount of the glycine is 13.4% by mass based on the total mass of the cosmetic,
an amount of the Na citrate is 10.4% by mass based on the total mass of the cosmetic,
an amount of the alanine is 7.6% by mass based on the total mass of the cosmetic,
an amount of the 2Na succinate is 7.47% by mass based on the total mass of the cosmetic, and
an amount of the Na sulfate is 4% by mass based on the total mass of the cosmetic.

The cosmetic according to the present invention can comprise 0.01% by mass of each of all of histidine, proline, isoleucine, serine, valine, aspartic acid, threonine, phenylalanine, arginine, PCA, PCA-Na, Na lactate, and water based on the total mass of the cosmetic.

Another cosmetic composition according to the present invention is a cosmetic composition comprising
arginine,
Na citrate,
glycine,
alanine,
trehalose, and
2Na succinate, wherein
an amount of the arginine is 72.46% by mass based on the total mass of the composition,
an amount of the Na citrate is 8.30% by mass based on the total mass of the composition,
an amount of the glycine is 4.81% by mass based on the total mass of the composition,
an amount of the alanine is 4.81% by mass based on the total mass of the composition,
an amount of the trehalose is 4.81% by mass based on the total mass of the composition, and
an amount of the 2Na succinate is 4.81% by mass based on the total mass of the composition.

Another cosmetic according to the present invention can be formed by incorporating one or more members selected from histidine, proline, isoleucine, serine, valine, aspartic acid, threonine, phenylalanine, PCA, PCA-Na, Na lactate, and water in the cosmetic composition according to claim 5.

Another cosmetic according to the present invention can be configured such that
an amount of the arginine is 72.38% by mass based on the total mass of the cosmetic,
an amount of the Na citrate is 8.3% by mass based on the total mass of the cosmetic,
an amount of the glycine is 4.8% by mass based on the total mass of the cosmetic,
an amount of the alanine is 4.8% by mass based on the total mass of the cosmetic,
an amount of the trehalose is 4.8% by mass based on the total mass of the cosmetic, and
an amount of the 2Na succinate is 4.8% by mass based on the total mass of the cosmetic.

Another cosmetic according to the present invention can comprise 0.01% by mass of each of all of histidine, proline, isoleucine, serine, valine, aspartic acid, threonine, phenylalanine, PCA, PCA-Na, Na lactate, and water based on the total mass of the cosmetic.

Another cosmetic composition according to the present invention is a cosmetic composition comprising
tartaric acid,
glycine,
Na citrate,
alanine,
2Na succinate,
arginine, and
Na sulfate, wherein
an amount of the tartaric acid is 28.04% by mass based on the total mass of the composition,
an amount of the glycine is 21.73% by mass based on the total mass of the composition,
an amount of the Na citrate is 17.02% by mass based on the total mass of the composition,
an amount of the alanine is 12.01% by mass based on the total mass of the composition,
an amount of the 2Na succinate is 12.01% by mass based on the total mass of the composition,
an amount of the arginine is 5.19% by mass based on the total mass of the composition, and
an amount of the Na sulfate is 4.00% by mass based on the total mass of the composition.

Another cosmetic according to the present invention can be formed by incorporating one or more members selected from histidine, proline, isoleucine, serine, valine, aspartic acid, threonine, phenylalanine, PCA, PCA-Na, Na lactate, and water in the cosmetic composition according to claim 9.

Another cosmetic according to the present invention can be configured such that
an amount of the tartaric acid is 28% by mass based on the total mass of the cosmetic,
an amount of the glycine is 21.7% by mass based on the total mass of the cosmetic,
an amount of the Na citrate is 17% by mass based on the total mass of the cosmetic,
an amount of the alanine is 12% by mass based on the total mass of the cosmetic,
an amount of the 2Na succinate is 12% by mass based on the total mass of the cosmetic,
an amount of the arginine is 5.18% by mass based on the total mass of the cosmetic, and
an amount of the Na sulfate is 4% by mass based on the total mass of the cosmetic.

Another cosmetic according to the present invention can comprise 0.01% by mass of each of all of histidine, proline, isoleucine, serine, valine, aspartic acid, threonine, phenylalanine, PCA, PCA-Na, Na lactate, and water based on the total mass of the cosmetic.

Still another cosmetic composition according to the present invention is a cosmetic composition comprising
glycine,
Na citrate,
arginine,
alanine,
2Na succinate, and
tartaric acid, wherein
an amount of the glycine is 25.03% by mass based on the total mass of the composition,
an amount of the Na citrate is 19.22% by mass based on the total mass of the composition,
an amount of the arginine is 16.72% by mass based on the total mass of the composition,
an amount of the alanine is 13.42% by mass based on the total mass of the composition,
an amount of the 2Na succinate is 13.42% by mass based on the total mass of the composition, and
an amount of the tartaric acid is 12.19% by mass based on the total mass of the composition.

Still another cosmetic according to the present invention can be formed by incorporating one or more members selected from histidine, proline, isoleucine, serine, valine, aspartic acid, threonine, phenylalanine, PCA, PCA-Na, Na lactate, and water in the cosmetic composition according to claim 13.

Still another cosmetic according to the present invention can be configured such that
an amount of the glycine is 25% by mass based on the total mass of the cosmetic,
an amount of the Na citrate is 19.2% by mass based on the total mass of the cosmetic,
an amount of the arginine is 16.7% by mass based on the total mass of the cosmetic,
an amount of the alanine is 13.4% by mass based on the total mass of the cosmetic,
an amount of the 2Na succinate is 13.4% by mass based on the total mass of the cosmetic, and
an amount of the tartaric acid is 12.18% by mass based on the total mass of the cosmetic.

Still another cosmetic according to the present invention can comprise 0.01% by mass of each of all of histidine, proline, isoleucine, serine, valine, aspartic acid, threonine, phenylalanine, PCA, PCA-Na, Na lactate, and water based on the total mass of the cosmetic.

The present invention includes a method for applying a cosmetic composition, the method comprising applying the cosmetic composition according to claim 1, 5, 9 or 13 to hair, the skin, hair of the head and/or the scalp.

The present invention includes a method for applying a cosmetic, the method comprising applying the cosmetic according to any one of claims 2 to 4, 6 to 8, 10 to 12, and 14 to 16 to hair, the skin, hair of the head and/or the scalp.

The present invention can provide a cosmetic composition having a novel formulation of components that can exert a high improving effect on the texture of hair of the head or the scalp, a cosmetic comprising this cosmetic composition, and a method for applying this cosmetic composition or cosmetic. The present invention can also provide a cosmetic composition that meets the laws and standards of foreign countries other than Japan and thus is usable in the world, a cosmetic comprising this cosmetic composition, and a method for applying this cosmetic composition or cosmetic.

### BRIEF DESCRIPTION OF THE DRAWINGS

FIG. 1 is a diagram showing the component ratios in Embodiment 1 which is an exemplary embodiment that can be adopted by the cosmetic composition according to the present invention;

FIG. 2 is a diagram showing Examples 1-1 to 1-4 which are specific examples of the cosmetic composition according to Embodiment 1 shown in FIG. 1;

FIG. 3 is a diagram showing the component ratios of the cosmetic according to Embodiment 1 that can be formed by adding predetermined component(s) to the cosmetic composition according to Embodiment 1 shown in FIG. 1;

FIG. 4 is a diagram showing Examples 1-1 to 1-4 which are specific examples of the cosmetic according to Embodiment 1 shown in FIG. 3;

FIG. 5 is a diagram showing the component ratios in Embodiment 2 which is an exemplary embodiment that can be adopted by the cosmetic composition according to the present invention;

FIG. 6 is a diagram showing Examples 2-1 to 2-4 which are specific examples of the cosmetic composition according to Embodiment 2 shown in FIG. 5;

FIG. 7 is a diagram showing the component ratios of the cosmetic according to Embodiment 2 that can be formed by adding predetermined component(s) to the cosmetic composition according to Embodiment 2 shown in FIG. 5;

FIG. 8 is a diagram showing Examples 2-1 to 2-4 which are specific examples of the cosmetic according to Embodiment 2 shown in FIG. 7;

FIG. 9 is a diagram showing the component ratios in Embodiment 3 which is an exemplary embodiment that can be adopted by the cosmetic composition according to the present invention;

FIG. 10 is a diagram showing Examples 3-1 to 3-4 which are specific examples of the cosmetic composition according to Embodiment 3 shown in FIG. 9;

FIG. 11 is a diagram showing the component ratios of the cosmetic according to Embodiment 3 that can be formed by adding predetermined component(s) to the cosmetic composition according to Embodiment 3 shown in FIG. 9;

FIG. 12 is a diagram showing Examples 3-1 to 3-4 which are specific examples of the cosmetic according to Embodiment 3 shown in FIG. 11;

FIG. 13 is a diagram showing the component ratios in Embodiment 4 which is an exemplary embodiment that can be adopted by the cosmetic composition according to the present invention;

FIG. 14 is a diagram showing Examples 4-1 to 4-4 which are specific examples of the cosmetic composition according to Embodiment 4 shown in FIG. 13;

FIG. 15 is a diagram showing the component ratios of the cosmetic according to Embodiment 4 that can be formed by adding predetermined component(s) to the cosmetic composition according to Embodiment 4 shown in FIG. 13; and

FIG. 16 is a diagram showing Examples 4-1 to 4-4 which are specific examples of the cosmetic according to Embodiment 4 shown in FIG. 15.

### DETAILED DESCRIPTION OF THE PREFERRED EMBODIMENTS

The present inventor has conducted diligent studies to find a cosmetic composition having a novel formulation of components that can exert a high improving effect on the texture of hair of the head, the scalp, or the like by examining various components and applying them to hair, the skin, hair of the head and/or the scalp. As a result, the present inventor has completed the present invention by finding that no nucleic acid heretofore known is used and tartaric acid and glycine and components such as, Na citrate (sodium citrate, hereafter referred to as “Na citrate”), arginine, alanine, 2Na succinate (disodium succinate, hereafter referred to as “2Na succinate”), and trehalose are contained while combinations and ratios of these substance components added are optimized, whereby the resulting cosmetic composition provides resilience or gloss to hair of the head, the scalp, hair, or the skin and is capable of exerting an effect such as improvement in hair cuticle or improvement in moisturizing effect, whitening effect, or combability of hair.

Hereinafter, the preferred embodiments of the present invention will be described with reference to the drawings. The following embodiments do not limit the invention according to each claim, and all combinations of features described in the embodiments are not always essential for the solution of the invention.

### [Cosmetic Composition of Embodiment 1]

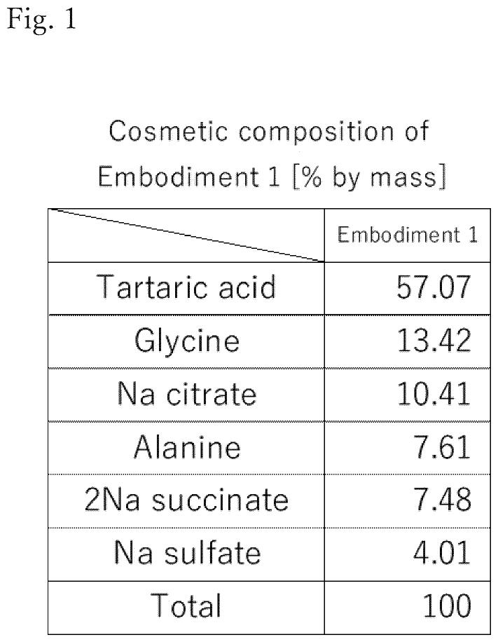

FIG. 1 is a diagram showing the component ratios in Embodiment 1 which is an exemplary embodiment that can be adopted by the cosmetic composition according to the present invention.

The cosmetic composition of Embodiment 1 is a cosmetic composition comprising
tartaric acid,
glycine,
Na citrate,
alanine,
2Na succinate, and
Na sulfate (sodium sulfate, hereafter referred to as “Na sulfate”), wherein
the amount of the tartaric acid is 57.07% by mass based on the total mass of the composition,
the amount of the glycine is 13.42% by mass based on the total mass of the composition,
the amount of the Na citrate is 10.41% by mass based on the total mass of the composition,
the amount of the alanine is 7.61% by mass based on the total mass of the composition,
the amount of the 2Na succinate is 7.48% by mass based on the total mass of the composition, and
the amount of the Na sulfate is 4.01% by mass based on the total mass of the composition.

The tartaric acid is a dicarboxylic acid and can fulfil a function of protecting hair through adsorption to fibrils of hair. The tartaric acid also functions as a substance that exerts an effect of lowing a pH value.

Each of the glycine and the alanine is a non-essential amino acid and is a substance necessary for repairing hair and thereby softening hair or imparting soft finish to the texture of hair. The glycine and the alanine each have the effect of cross-linking amino acids and can produce the effect of strengthening hair, i.e., enhancing the bearing force of hair to render the hair firm.

The citric acid is a tricarboxylic acid and can exert the effect of protecting hair so as not to damage fibrils of hair. The citric acid exerts a chelating effect or fulfils a function as a sequestering agent and the citric acid can therefore also produce the effect of stabilizing the cosmetic composition. The citric acid further functions as a substance that adjusts pH. Na citrate is used as the citric acid of Embodiment 1, and different citric acid, i.e., monosodium citrate, disodium citrate or trisodium citrate, may be used. In short, the Na citrate of Embodiment 1 can include monosodium citrate, disodium citrate or trisodium citrate.

The 2Na succinate is a dicarboxylic acid and is a substance having a function of protecting fibrils of hair. The 2Na succinate also functions as a substance that adjusts pH.

The Na sulfate functions as a surfactant. The Na sulfate possesses properties of a hydrophilic group and a lipophilic group of a surfactant and the Na sulfate therefore has excellent sebum stain-cleansing power and can fulfil a function of cleaning hair and/or the scalp.

The cosmetic composition of Embodiment 1 has the substances having the characteristics mentioned above at the component blending ratio described above and can thereby exert a high improving effect on the texture of hair of the head or the scalp. Particularly, the cosmetic composition of Embodiment 1 has the effect of largely improving the effect of a dicarboxylic acid, i.e., the effect of protecting hair through adsorption to fibrils of hair because the amount of the tartaric acid is set to as high as 57.07% by mass based on the total mass of the composition. The cosmetic composition of Embodiment 1 also has a sterilizing effect or a peeling effect on hair or the skin because its pH value is acidic pH of 2.5 to 3.0. This cosmetic composition can produce, for example, the effect of softening and lysing old dead cells on the surface of the skin or promoting the metabolism of the body.

### [Examples of Cosmetic Composition of Embodiment 1]

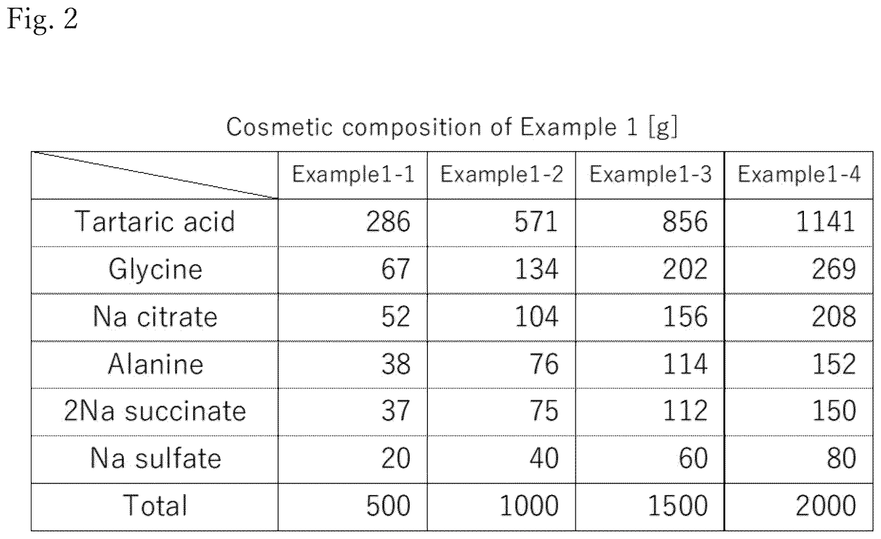

The preferred embodiments of the cosmetic composition of Embodiment 1 are described above. Next, specific examples of the cosmetic composition of Embodiment 1 mentioned above will be given with reference to FIG. 2. FIG. 2 is a diagram showing Examples 1-1 to 1-4 which are specific examples of the cosmetic composition according to Embodiment 1 shown in FIG. 1.

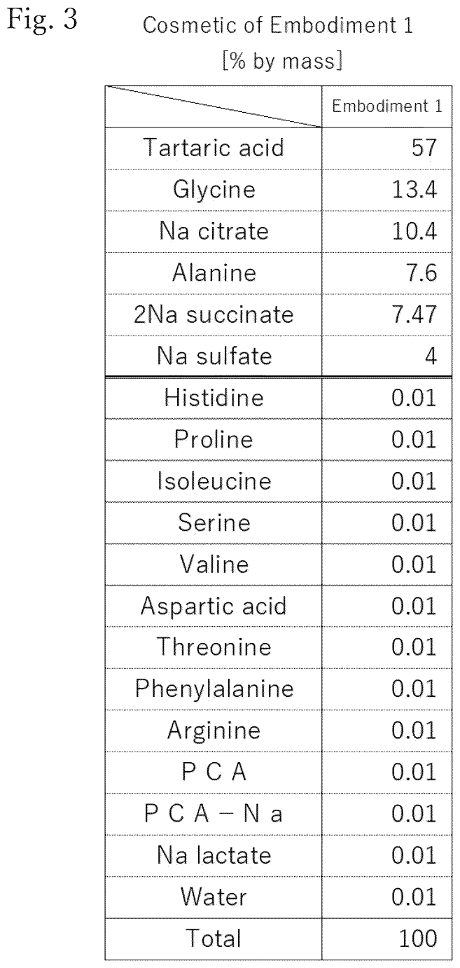

FIG. 2 illustrates specific amounts of the components for forming a cosmetic composition having component ratios according to the inventive formulation shown in Embodiment 1 mentioned above. For example, FIG. 2 indicates a specific quantity of each component by the number of grams as Example 1-1 when 500 g in total of the cosmetic composition according to Embodiment 1 mentioned above is formed. Likewise, Example 1-2 in FIG. 2 depicts a specific quantity of each component when 1000 g in total of the cosmetic composition according to Embodiment 1 mentioned above is formed. Example 1-3 in FIG. 3 depicts a specific quantity of each component when 1500 g in total of the cosmetic composition according to Embodiment 1 mentioned above is formed. Example 1-4 in FIG. 3 depicts a specific quantity of each component when 2000 g in total of the cosmetic composition according to Embodiment 1 mentioned above is formed.

The specific amounts of the components in the cosmetic composition of the present invention will become obvious with reference to Examples 1-1 to 1-4 illustrated in FIG. 2. The cosmetic composition according to each example can also exert the working effects described for the embodiment mentioned above, irrespective of the number of grams of the total quantity, as long as the cosmetic composition has the component ratios shown in FIG. 1, of Embodiment 1 mentioned above.

The cosmetic of the present invention can be formed by further adding an additional component to the cosmetic composition having the component ratios according to the formulation of the invention shown in Embodiment 1 mentioned above.

### [Cosmetic of Embodiment 1]

Specifically, the cosmetic of Embodiment 1 having the efficacy of the cosmetic composition according to Embodiment 1 can be formed by adding one or more members selected from histidine, proline, isoleucine, serine, valine, aspartic acid, threonine, phenylalanine, arginine, PCA (Pyrrolidone Carboxylic Acid), PCA-Na (sodium pyrrolidonecarboxylate), Na lactate (sodium lactate, hereafter referred to as “Na lactate”), and water to the cosmetic composition of Embodiment 1 mentioned above. FIG. 3 shows the component ratios of the cosmetic of Embodiment 1 which is configured so as to comprise the cosmetic composition according to Embodiment 1. FIG. 3 is a diagram showing the component ratios of the cosmetic according to Embodiment 1 that can be formed by adding predetermined component(s) to the cosmetic composition according to Embodiment 1 shown in FIG. 1.

As shown in FIG. 3, the component ratios of the cosmetic according to Embodiment 1 are such that
the amount of the tartaric acid is 57% by mass based on the total mass of the cosmetic,
the amount of the glycine is 13.4% by mass based on the total mass of the cosmetic,
the amount of the Na citrate is 10.4% by mass based on the total mass of the cosmetic,
the amount of the alanine is 7.6% by mass based on the total mass of the cosmetic,
the amount of the 2Na succinate is 7.47% by mass based on the total mass of the cosmetic, and
the amount of the Na sulfate is 4% by mass based on the total mass of the cosmetic, and
0.01% by mass of each of all of histidine, proline, isoleucine, serine, valine, aspartic acid, threonine, phenylalanine, arginine, PCA, PCA-Na, Na lactate, and water is further contained based on the total mass of the cosmetic. The cosmetic of Embodiment 1 can be configured by these component ratios and therefore produced as a cosmetic having preferred efficacy.

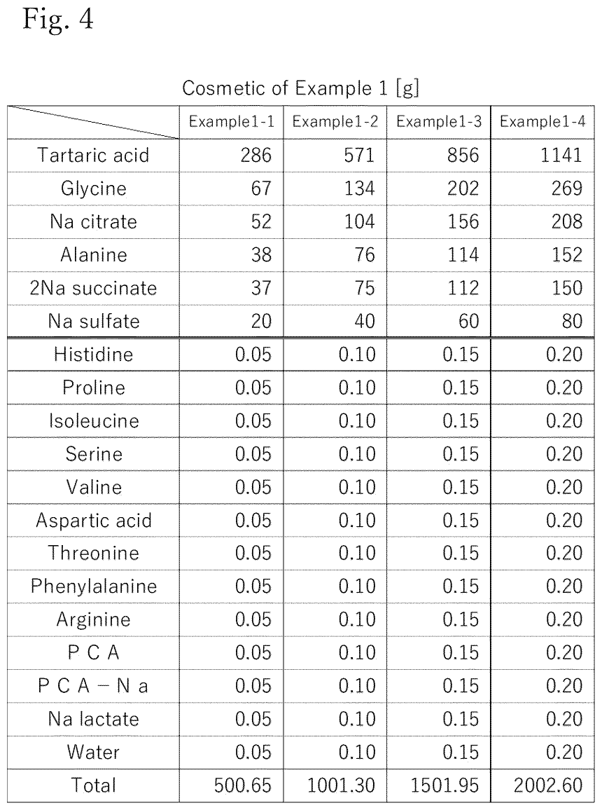

Next, specific examples of the cosmetic of Embodiment 1 will be given with reference to FIG. 4. FIG. 4 is a diagram showing Examples 1-1 to 1-4 which are specific examples of the cosmetic according to Embodiment 1 shown in FIG. 3.

### [Examples of Cosmetic of Embodiment 1]

FIG. 4 illustrates specific amounts of the components for forming a cosmetic having component ratios according to the inventive formulation shown in Embodiment 1 mentioned above. For example, FIG. 4 indicates a specific quantity by the number of grams as Example 1-1, of a cosmetic formed by forming 500 g of the cosmetic composition according to Embodiment 1 shown in FIG. 2 and adding all of histidine, proline, isoleucine, serine, valine, aspartic acid, threonine, phenylalanine, arginine, PCA, PCA-Na, Na lactate, and water to the cosmetic composition so that the component ratios in FIG. 3 are met. Likewise, Example 1-2 in FIG. 4 depicts a specific quantity by a specific number of grams, of a cosmetic formed by forming 1000 g of the cosmetic composition according to Embodiment 1 mentioned above and adding all of histidine, proline, isoleucine, serine, valine, aspartic acid, threonine, phenylalanine, arginine, PCA, PCA-Na, Na lactate, and water to the cosmetic composition so that the component ratios in FIG. 3 are met. Likewise, Example 1-3 in FIG. 4 depicts a specific quantity by a specific number of grams, of a cosmetic formed by forming 1500 g of the cosmetic composition according to Embodiment 1 mentioned above and adding all of histidine, proline, isoleucine, serine, valine, aspartic acid, threonine, phenylalanine, arginine, PCA, PCA-Na, Na lactate, and water to the cosmetic composition so that the component ratios in FIG. 3 are met. Likewise, Example 1-4 in FIG. 4 depicts a specific quantity by a specific number of grams, of a cosmetic formed by forming 2000 g of the cosmetic composition according to Embodiment 1 mentioned above and adding all of histidine, proline, isoleucine, serine, valine, aspartic acid, threonine, phenylalanine, arginine, PCA, PCA-Na, Na lactate, and water to the cosmetic composition so that the component ratios in FIG. 3 are met.

The specific amounts of the components in the cosmetic of Embodiment 1 will become obvious with reference to Examples 1-1 to 1-4 illustrated in FIG. 4. The cosmetic according to each example can also exert the working effects described for the embodiment mentioned above, irrespective of the number of grams of the total quantity, as long as the cosmetic has the component ratios shown in FIG. 3, of Embodiment 1 mentioned above.

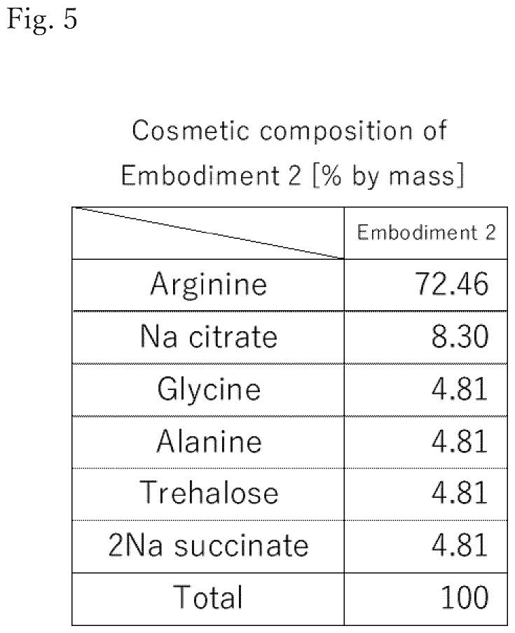

The cosmetic composition according to Embodiment 1 and the cosmetic using this cosmetic composition are described with reference to FIG. 1 to FIG. 4. Next, the cosmetic composition according to Embodiment 2 and the cosmetic using this cosmetic composition will be described with reference to FIG. 5 to FIG. 8.

### [Cosmetic Composition of Embodiment 2]

FIG. 5 is a diagram showing the component ratios in Embodiment 2 which is an exemplary embodiment that can be adopted by the cosmetic composition according to the present invention.

The cosmetic composition of Embodiment 2 is a cosmetic composition comprising
arginine,
Na citrate,
glycine,
alanine,
trehalose, and
2Na succinate, wherein
the amount of the arginine is 72.46% by mass based on the total mass of the composition,
the amount of the Na citrate is 8.30% by mass based on the total mass of the composition,
the amount of the glycine is 4.81% by mass based on the total mass of the composition,
the amount of the alanine is 4.81% by mass based on the total mass of the composition,
the amount of the trehalose is 4.81% by mass based on the total mass of the composition, and
the amount of the 2Na succinate is 4.81% by mass based on the total mass of the composition.

The arginine is an amino acid also contained in hair and is a substance for use as an alkali agent. The arginine is also a substance having the effect of promoting the secretion of growth hormone, the effect of enhancing immune strength, and the effect of detoxicating ammonia. The arginine further has the force to largely open hair cuticle and therefore allows the cosmetic composition of Embodiment 2 to penetrate into hair, where the cosmetic composition can exert the effect of improving the texture of hair.

The citric acid is a tricarboxylic acid and can exert the effect of protecting hair so as not to damage fibrils of hair. The citric acid exerts a chelating effect or fulfils a function as a sequestering agent and the citric acid can therefore also produce the effect of stabilizing the cosmetic composition. The citric acid further functions as a substance that adjusts pH. Na citrate is used as the citric acid of Embodiment 2, and different citric acid, i.e., monosodium citrate, disodium citrate or trisodium citrate, may be used. In short, the Na citrate of Embodiment 2 can include monosodium citrate, disodium citrate or trisodium citrate.

Each of the glycine and the alanine is a non-essential amino acid and is a substance necessary for repairing hair and thereby softening hair or imparting soft finish to the texture of hair. The glycine and the alanine each have the effect of cross-linking amino acids and can produce the effect of strengthening hair, i.e., enhancing the bearing force of hair to render the hair firm.

The trehalose has the effect of preventing the disruption of proteins contained in hair and can exert the effect of enhancing hair's ability to retain moisture.

The 2Na succinate is a dicarboxylic acid and is a substance having a function of protecting fibrils of hair. The 2Na succinate functions as a substance that adjusts pH.

The cosmetic composition of Embodiment 2 has the substances having the characteristics mentioned above at the component blending ratio described above and can thereby exert a high improving effect on the texture of hair of the head or the scalp. Particularly, the cosmetic composition of Embodiment 2 has the effect of an alkali agent, i.e., the effect of promoting the secretion of growth hormone and then enhancing immune strength and also detoxicating ammonia because the amount of the arginine is set to as high as 72.46% by mass based on the total mass of the composition. The cosmetic composition of Embodiment 2 can improve the texture of hair by the effect of the arginine which largely opens hair cuticle and allows the cosmetic composition of Embodiment 2 to penetrate into hair.

### [Examples of Cosmetic Composition of Embodiment 2]

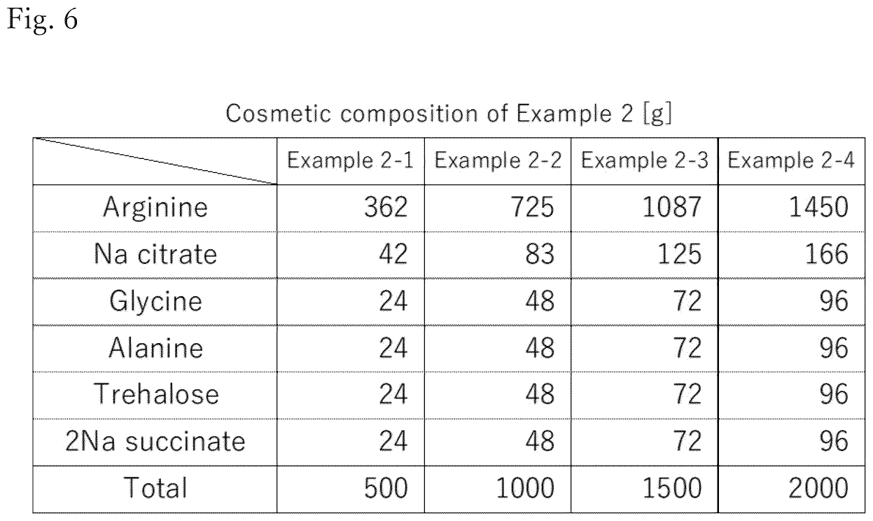

The preferred embodiments of the cosmetic composition of Embodiment 2 are described above. Next, specific examples of the cosmetic composition of Embodiment 2 mentioned above will be given with reference to FIG. 6. FIG. 6 is a diagram showing Examples 2-1 to 2-4 which are specific examples of the cosmetic composition according to Embodiment 2 shown in FIG. 5.

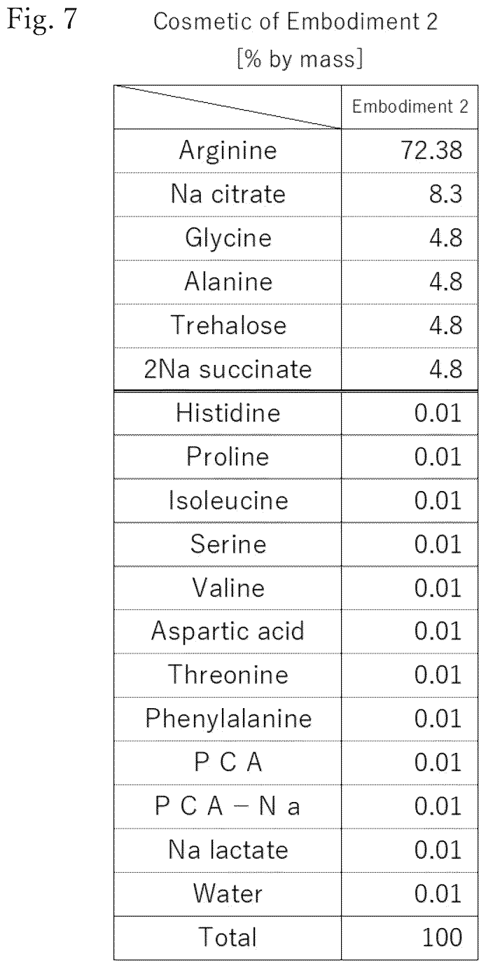

FIG. 6 illustrates specific amounts of the components for forming a cosmetic composition having component ratios according to the inventive formulation shown in Embodiment 2 mentioned above. For example, FIG. 6 indicates a specific quantity of each component by the number of grams as Example 2-1 when 500 g in total of the cosmetic composition according to Embodiment 2 mentioned above is formed. Likewise, Example 2-2 in FIG. 6 depicts a specific quantity of each component when 1000 g in total of the cosmetic composition according to Embodiment 2 mentioned above is formed. Example 2-3 in FIG. 7 depicts a specific quantity of each component when 1500 g in total of the cosmetic composition according to Embodiment 2 mentioned above is formed. Example 2-4 in FIG. 7 depicts a specific quantity of each component when 2000 g in total of the cosmetic composition according to Embodiment 2 mentioned above is formed.

The specific amounts of the components in the cosmetic composition of the present invention will become obvious with reference to Examples 2-1 to 2-4 illustrated in FIG. 6. The cosmetic composition according to each example can also exert the working effects described for the embodiment mentioned above, irrespective of the number of grams of the total quantity, as long as the cosmetic composition has the component ratios shown in FIG. 5, of Embodiment 2 mentioned above.

The cosmetic of the present invention can be formed by further adding an additional component to the cosmetic composition having the component ratios according to the formulation of the invention shown in Embodiment 2 mentioned above.

### [Cosmetic of Embodiment 2]

Specifically, the cosmetic of Embodiment 2 having the efficacy of the cosmetic composition according to Embodiment 2 can be formed by adding one or more members selected from histidine, proline, isoleucine, serine, valine, aspartic acid, threonine, phenylalanine, PCA, PCA-Na, Na lactate, and water to the cosmetic composition of Embodiment 2 mentioned above. FIG. 7 shows the component ratios of the cosmetic of Embodiment 2 which is configured so as to comprise the cosmetic composition according to Embodiment 2. FIG. 7 is a diagram showing the component ratios of the cosmetic according to Embodiment 2 that can be formed by adding predetermined component(s) to the cosmetic composition according to Embodiment 2 shown in FIG. 5.

As shown in FIG. 7, the component ratios of the cosmetic according to Embodiment 2 are such that
the amount of the arginine is 72.38% by mass based on the total mass of the cosmetic,
the amount of the Na citrate is 8.3% by mass based on the total mass of the cosmetic,
the amount of the glycine is 4.8% by mass based on the total mass of the cosmetic,
the amount of the alanine is 4.8% by mass based on the total mass of the cosmetic,
the amount of the trehalose is 4.8% by mass based on the total mass of the cosmetic, and
the amount of the 2Na succinate is 4.8% by mass based on the total mass of the cosmetic, and
0.01% by mass of each of all of histidine, proline, isoleucine, serine, valine, aspartic acid, threonine, phenylalanine, PCA, PCA-Na, Na lactate, and water is further contained based on the total mass of the cosmetic. The cosmetic of Embodiment 2 can be configured by these component ratios and therefore produced as a cosmetic having preferred efficacy.

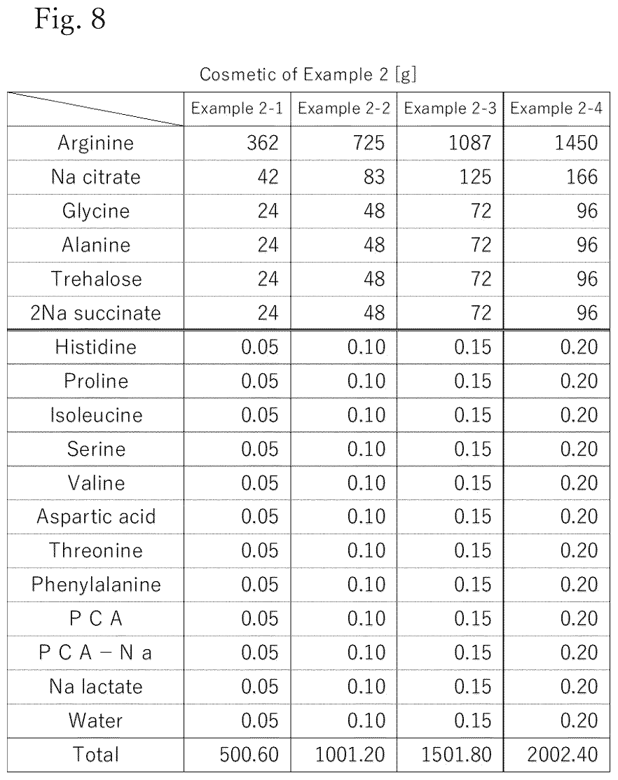

Next, specific examples of the cosmetic of Embodiment 2 will be given with reference to FIG. 8. FIG. 8 is a diagram showing Examples 2-1 to 2-4 which are specific examples of the cosmetic according to Embodiment 2 shown in FIG. 7.

### [Examples of Cosmetic of Embodiment 2]

FIG. 8 illustrates specific amounts of the components for forming a cosmetic having component ratios according to the inventive formulation shown in Embodiment 2 mentioned above. For example, FIG. 8 indicates a specific quantity by the number of grams as Example 2-1, of a cosmetic formed by forming 500 g of the cosmetic composition according to Embodiment 2 shown in FIG. 6 and adding all of histidine, proline, isoleucine, serine, valine, aspartic acid, threonine, phenylalanine, PCA, PCA-Na, Na lactate, and water to the cosmetic composition so that the component ratios in FIG. 7 are met. Likewise, Example 2-2 in FIG. 8 depicts a specific quantity by a specific number of grams, of a cosmetic formed by forming 1000 g of the cosmetic composition according to Embodiment 2 mentioned above and adding all of histidine, proline, isoleucine, serine, valine, aspartic acid, threonine, phenylalanine, PCA, PCA-Na, Na lactate, and water to the cosmetic composition so that the component ratios in FIG. 7 are met. Likewise, Example 2-3 in FIG. 8 depicts a specific quantity by a specific number of grams, of a cosmetic formed by forming 1500 g of the cosmetic composition according to Embodiment 2 mentioned above and adding all of histidine, proline, isoleucine, serine, valine, aspartic acid, threonine, phenylalanine, PCA, PCA-Na, Na lactate, and water to the cosmetic composition so that the component ratios in FIG. 7 are met. Likewise, Example 2-4 in FIG. 8 depicts a specific quantity by a specific number of grams, of a cosmetic formed by forming 2000 g of the cosmetic composition according to Embodiment 2 mentioned above and adding all of histidine, proline, isoleucine, serine, valine, aspartic acid, threonine, phenylalanine, PCA, PCA-Na, Na lactate, and water to the cosmetic composition so that the component ratios in FIG. 7 are met.

The specific amounts of the components in the cosmetic of Embodiment 2 will become obvious with reference to Examples 2-1 to 2-4 illustrated in FIG. 8. The cosmetic according to each example can also exert the working effects described for the embodiment mentioned above, irrespective of the number of grams of the total quantity, as long as the cosmetic has the component ratios shown in FIG. 7, of Embodiment 2 mentioned above.

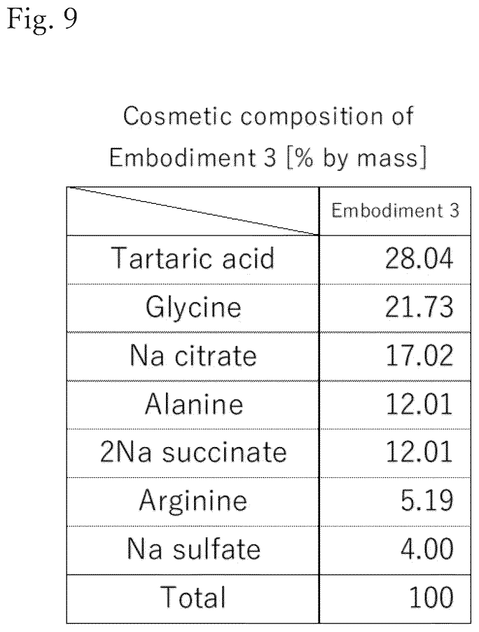

The cosmetic composition according to Embodiment 2 and the cosmetic using this cosmetic composition are described with reference to FIG. 5 to FIG. 8. Next, the cosmetic composition according to Embodiment 3 and the cosmetic using this cosmetic composition will be described with reference to FIG. 9 to FIG. 12.

### [Cosmetic Composition of Embodiment 3]

FIG. 9 is a diagram showing the component ratios in Embodiment 3 which is an exemplary embodiment that can be adopted by the cosmetic composition according to the present invention.

The cosmetic composition of Embodiment 3 is a cosmetic composition comprising
tartaric acid,
glycine,
Na citrate,
alanine,
2Na succinate,
arginine, and
Na sulfate, wherein
the amount of the tartaric acid is 28.04% by mass based on the total mass of the composition,
the amount of the glycine is 21.73% by mass based on the total mass of the composition,
the amount of the Na citrate is 17.02% by mass based on the total mass of the composition,
the amount of the alanine is 12.01% by mass based on the total mass of the composition,
the amount of the 2Na succinate is 12.01% by mass based on the total mass of the composition,
the amount of the arginine is 5.19% by mass based on the total mass of the composition, and
the amount of the Na sulfate is 4.00% by mass based on the total mass of the composition.

The tartaric acid is a dicarboxylic acid and can fulfil a function of protecting hair through adsorption to fibrils of hair. The tartaric acid also functions as a substance that exerts an effect of lowing a pH value.

Each of the glycine and the alanine is a non-essential amino acid and is a substance necessary for repairing hair and thereby softening hair or imparting soft finish to the texture of hair. The glycine and the alanine each have the effect of cross-linking amino acids and can produce the effect of strengthening hair, i.e., enhancing the bearing force of hair to render the hair firm.

The citric acid is a tricarboxylic acid and can exert the effect of protecting hair so as not to damage fibrils of hair. The citric acid exerts a chelating effect or fulfils a function as a sequestering agent and the citric acid can therefore also produce the effect of stabilizing the cosmetic composition. The citric acid further functions as a substance that adjusts pH. Na citrate is used as the citric acid of Embodiment 3, and different citric acid, i.e., monosodium citrate, disodium citrate or trisodium citrate, may be used. In short, the citric acid (Na citrate) of Embodiment 3 can include monosodium citrate, disodium citrate or trisodium citrate.

The 2Na succinate is a dicarboxylic acid and is a substance having a function of protecting fibrils of hair. Succinic acid also functions as a substance that adjusts pH.

The arginine is an amino acid also contained in hair and is a substance for use as an alkali agent. The arginine is also a substance having the effect of promoting the secretion of growth hormone, the effect of enhancing immune strength, and the effect of detoxicating ammonia. The arginine further has the force to largely open hair cuticle and therefore allows the cosmetic composition of Embodiment 3 to penetrate into hair, where the cosmetic composition can exert the effect of improving the texture of hair.

The Na sulfate functions as a surfactant. The Na sulfate possesses properties of a hydrophilic group and a lipophilic group of a surfactant and the Na sulfate therefore has excellent sebum stain-cleansing power and can fulfil a function of cleaning hair and/or the scalp.

The cosmetic composition of Embodiment 3 has the substances having the characteristics mentioned above at the component blending ratio described above and can thereby exert a high improving effect on the texture of hair of the head or the scalp. Particularly, the cosmetic composition of Embodiment 3 comprises each of the components blended in a well-balanced manner and therefore can improve the texture of hair by allowing the efficacy of each of the components to be exerted.

### [Examples of Cosmetic Composition of Embodiment 3]

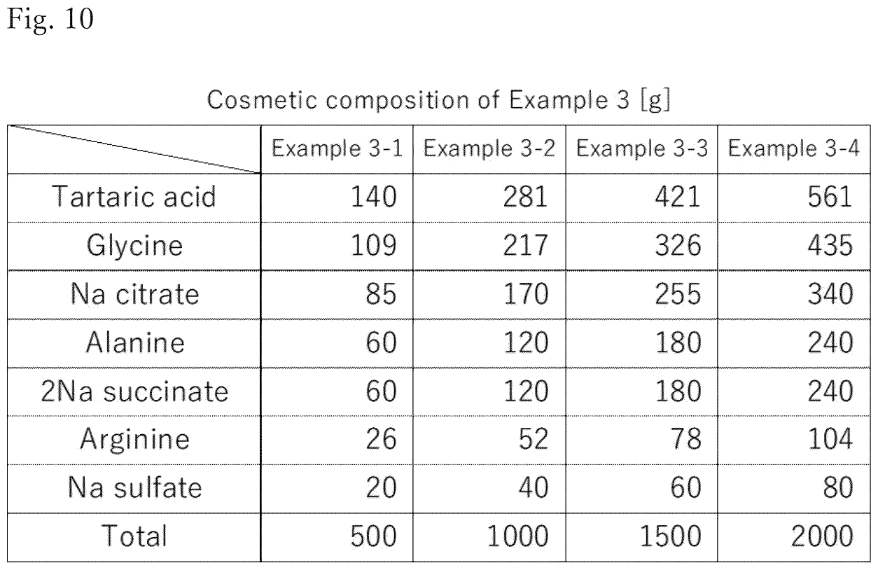

The preferred embodiments of the cosmetic composition of Embodiment 3 are described above. Next, specific examples of the cosmetic composition of Embodiment 3 mentioned above will be given with reference to FIG. 10. FIG. 10 is a diagram showing Examples 3-1 to 3-4 which are specific examples of the cosmetic composition according to Embodiment 3 shown in FIG. 9.

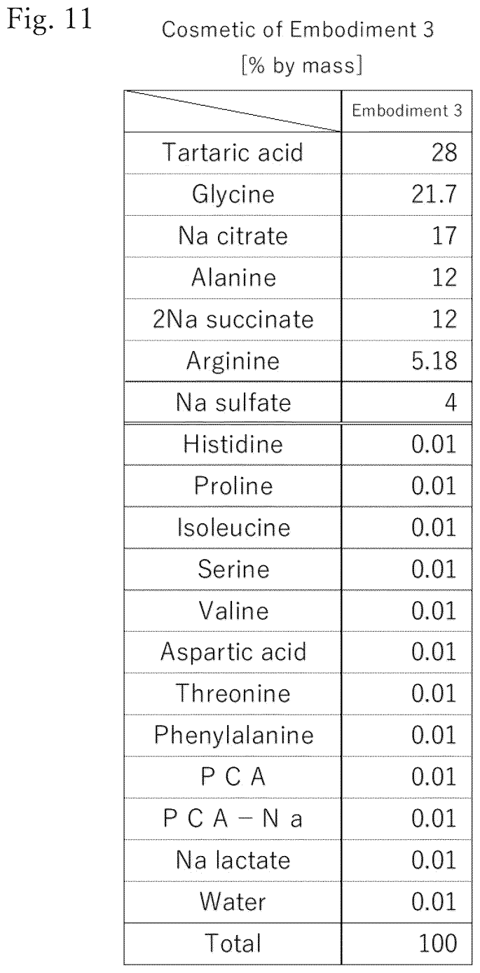

FIG. 10 illustrates specific amounts of the components for forming a cosmetic composition having component ratios according to the inventive formulation shown in Embodiment 3 mentioned above. For example, FIG. 10 indicates a specific quantity of each component by the number of grams as Example 3-1 when 500 g in total of the cosmetic composition according to Embodiment 3 mentioned above is formed. Likewise, Example 3-2 in FIG. 10 depicts a specific quantity of each component when 1000 g in total of the cosmetic composition according to Embodiment 3 mentioned above is formed. Example 3-3 in FIG. 11 depicts a specific quantity of each component when 1500 g in total of the cosmetic composition according to Embodiment 3 mentioned above is formed. Example 3-4 in FIG. 11 depicts a specific quantity of each component when 2000 g in total of the cosmetic composition according to Embodiment 3 mentioned above is formed.

The specific amounts of the components in the cosmetic composition of the present invention will become obvious with reference to Examples 3-1 to 3-4 illustrated in FIG. 10. The cosmetic composition according to each example can also exert the working effects described for the embodiment mentioned above, irrespective of the number of grams of the total quantity, as long as the cosmetic composition has the component ratios shown in FIG. 9, of Embodiment 3 mentioned above.

The cosmetic of the present invention can be formed by further adding an additional component to the cosmetic composition having the component ratios according to the formulation of the invention shown in Embodiment 3 mentioned above.

### [Cosmetic of Embodiment 3]

Specifically, the cosmetic of Embodiment 3 having the efficacy of the cosmetic composition according to Embodiment 3 can be formed by adding one or more members selected from histidine, proline, isoleucine, serine, valine, aspartic acid, threonine, phenylalanine, PCA, PCA-Na, Na lactate, and water to the cosmetic composition of Embodiment 3 mentioned above. FIG. 11 shows the component ratios of the cosmetic of Embodiment 3 which is configured so as to comprise the cosmetic composition according to Embodiment 3. FIG. 11 is a diagram showing the component ratios of the cosmetic according to Embodiment 3 that can be formed by adding predetermined component(s) to the cosmetic composition according to Embodiment 3 shown in FIG. 9.

As shown in FIG. 11, the component ratios of the cosmetic according to Embodiment 3 are such that
the amount of the tartaric acid is 28% by mass based on the total mass of the cosmetic,
the amount of the glycine is 21.7% by mass based on the total mass of the cosmetic,
the amount of the Na citrate is 17% by mass based on the total mass of the cosmetic,
the amount of the alanine is 12% by mass based on the total mass of the cosmetic,
the amount of the 2Na succinate is 12% by mass based on the total mass of the cosmetic,
the amount of the arginine is 5.18% by mass based on the total mass of the cosmetic, and
the amount of the Na sulfate is 4% by mass based on the total mass of the cosmetic, and
0.01% by mass of each of all of histidine, proline, isoleucine, serine, valine, aspartic acid, threonine, phenylalanine, PCA, PCA-Na, Na lactate, and water is further contained based on the total mass of the cosmetic. The cosmetic of Embodiment 3 can be configured by these component ratios and therefore produced as a cosmetic having preferred efficacy.

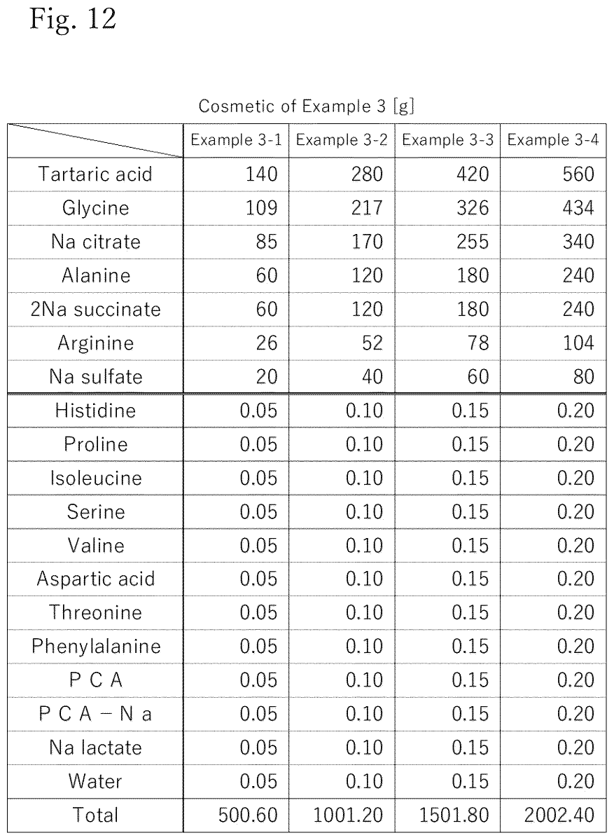

Next, specific examples of the cosmetic of Embodiment 3 will be given with reference to FIG. 12. FIG. 12 is a diagram showing Examples 3-1 to 3-4 which are specific examples of the cosmetic according to Embodiment 3 shown in FIG. 11.

### [Examples of Cosmetic of Embodiment 3]

FIG. 12 illustrates specific amounts of the components for forming a cosmetic having component ratios according to the inventive formulation shown in Embodiment 3 mentioned above. For example, FIG. 12 indicates a specific quantity by the number of grams as Example 3-1, of a cosmetic formed by forming 500 g of the cosmetic composition according to Embodiment 3 shown in FIG. 10 and adding all of histidine, proline, isoleucine, serine, valine, aspartic acid, threonine, phenylalanine, PCA, PCA-Na, Na lactate, and water to the cosmetic composition so that the component ratios in FIG. 11 are met. Likewise, Example 3-2 in FIG. 12 depicts a specific quantity by a specific number of grams, of a cosmetic formed by forming 1000 g of the cosmetic composition according to Embodiment 3 mentioned above and adding all of histidine, proline, isoleucine, serine, valine, aspartic acid, threonine, phenylalanine, PCA, PCA-Na, Na lactate, and water to the cosmetic composition so that the component ratios in FIG. 11 are met. Likewise, Example 3-3 in FIG. 12 depicts a specific quantity by a specific number of grams, of a cosmetic formed by forming 1500 g of the cosmetic composition according to Embodiment 3 mentioned above and adding all of histidine, proline, isoleucine, serine, valine, aspartic acid, threonine, phenylalanine, PCA, PCA-Na, Na lactate, and water to the cosmetic composition so that the component ratios in FIG. 11 are met. Likewise, Example 3-4 in FIG. 12 depicts a specific quantity by a specific number of grams, of a cosmetic formed by forming 2000 g of the cosmetic composition according to Embodiment 3 mentioned above and adding all of histidine, proline, isoleucine, serine, valine, aspartic acid, threonine, phenylalanine, PCA, PCA-Na, Na lactate, and water to the cosmetic composition so that the component ratios in FIG. 11 are met.

The specific amounts of the components in the cosmetic of Embodiment 3 will become obvious with reference to Examples 3-1 to 3-4 illustrated in FIG. 12. The cosmetic according to each example can also exert the working effects described for the embodiment mentioned above, irrespective of the number of grams of the total quantity, as long as the cosmetic has the component ratios shown in FIG. 11, of Embodiment 3 mentioned above.

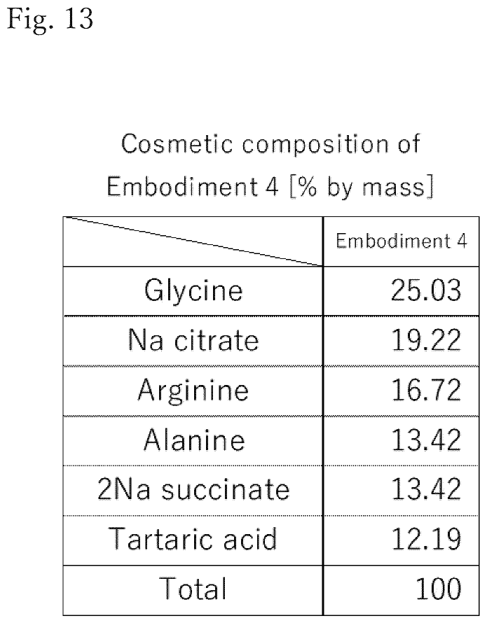

The cosmetic composition according to Embodiment 3 and the cosmetic using this cosmetic composition are described with reference to FIG. 9 to FIG. 12. Next, the cosmetic composition according to Embodiment 4 and the cosmetic using this cosmetic composition will be described with reference to FIG. 13 to FIG. 16.

### [Cosmetic Composition of Embodiment 4]

FIG. 13 is a diagram showing the component ratios in Embodiment 4 which is an exemplary embodiment that can be adopted by the cosmetic composition according to the present invention.

The cosmetic composition of Embodiment 4 is a cosmetic composition comprising
glycine,
Na citrate,
arginine,
alanine,
2Na succinate, and
tartaric acid, wherein
the amount of the glycine is 25.03% by mass based on the total mass of the composition,
the amount of the Na citrate is 19.22% by mass based on the total mass of the composition,
the amount of the arginine is 16.72% by mass based on the total mass of the composition,
the amount of the alanine is 13.42% by mass based on the total mass of the composition,
the amount of the 2Na succinate is 13.42% by mass based on the total mass of the composition, and
the amount of the tartaric acid is 12.19% by mass based on the total mass of the composition.

Each of the glycine and the alanine is a non-essential amino acid and is a substance necessary for repairing hair and thereby softening hair or imparting soft finish to the texture of hair. The glycine and the alanine each have the effect of cross-linking amino acids and can produce the effect of strengthening hair, i.e., enhancing the bearing force of hair to render the hair firm.

The citric acid is a tricarboxylic acid and can exert the effect of protecting hair so as not to damage fibrils of hair. The citric acid exerts a chelating effect or fulfils a function as a sequestering agent and the citric acid can therefore also produce the effect of stabilizing the cosmetic composition. The citric acid further functions as a substance that adjusts pH. Na citrate is used as the citric acid of Embodiment 4, and different citric acid, i.e., monosodium citrate, disodium citrate or trisodium citrate, may be used. In short, the citric acid (Na citrate) of Embodiment 4 can include monosodium citrate, disodium citrate or trisodium citrate.

The arginine is an amino acid also contained in hair and is a substance for use as an alkali agent. The arginine is also a substance having the effect of promoting the secretion of growth hormone, the effect of enhancing immune strength, and the effect of detoxicating ammonia. The arginine further has the force to largely open hair cuticle and therefore allows the cosmetic composition of Embodiment 4 to penetrate into hair, where the cosmetic composition can exert the effect of improving the texture of hair.

The 2Na succinate is a dicarboxylic acid and is a substance having a function of protecting fibrils of hair. Succinic acid also functions as a substance that adjusts pH.

The tartaric acid is a dicarboxylic acid and can fulfil a function of protecting hair through adsorption to fibrils of hair. The tartaric acid also functions as a substance that exerts an effect of lowing a pH value.

The cosmetic composition of Embodiment 4 has the substances having the characteristics mentioned above at the component blending ratio described above and can thereby exert a high improving effect on the texture of hair of the head or the scalp. Particularly, the cosmetic composition of Embodiment 4 comprises each of the components blended in a well-balanced manner and therefore can improve the texture of hair by allowing the efficacy of each of the components to be exerted.

### [Examples of Cosmetic Composition of Embodiment 4]

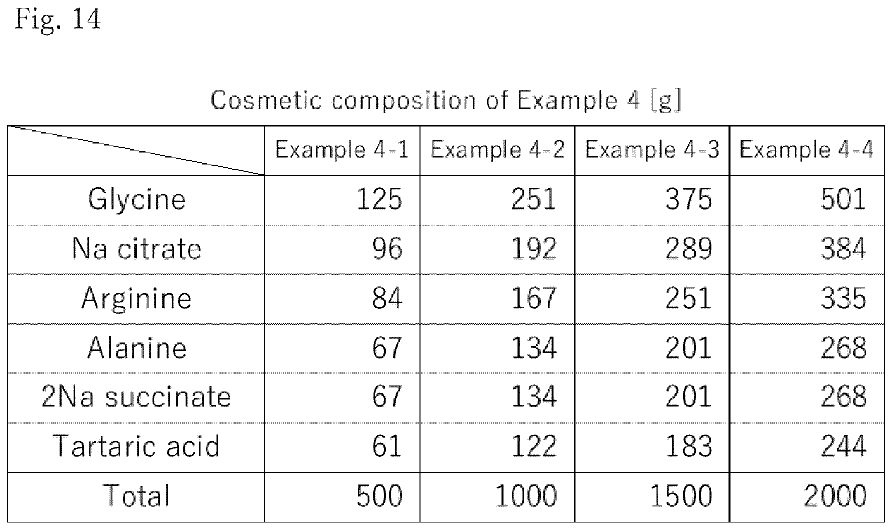

The preferred embodiments of the cosmetic composition of Embodiment 4 are described above. Next, specific examples of the cosmetic composition of Embodiment 4 mentioned above will be given with reference to FIG. 14. FIG. 14 is a diagram showing Examples 4-1 to 4-4 which are specific examples of the cosmetic composition according to Embodiment 4 shown in FIG. 13.

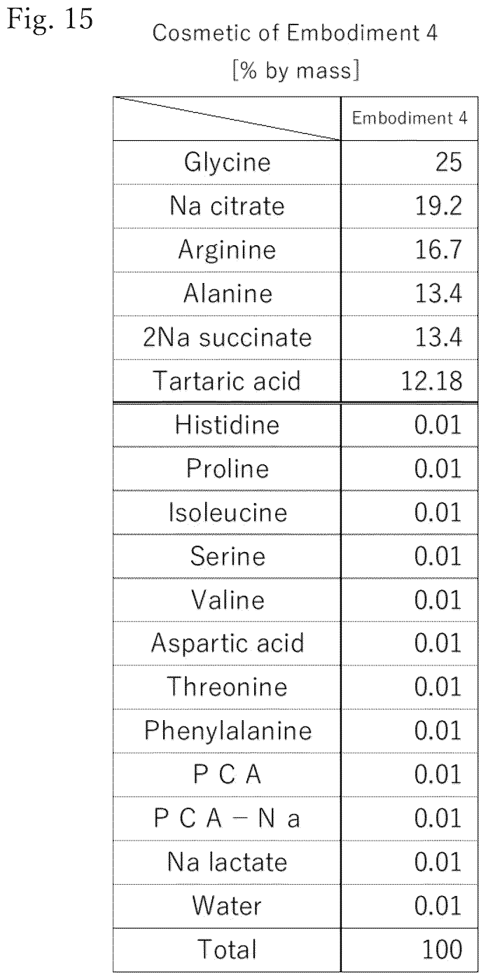

FIG. 14 illustrates specific amounts of the components for forming a cosmetic composition having component ratios according to the inventive formulation shown in Embodiment 4 mentioned above. For example, FIG. 14 indicates a specific quantity of each component by the number of grams as Example 4-1 when 500 g in total of the cosmetic composition according to Embodiment 4 mentioned above is formed. Likewise, Example 4-2 in FIG. 14 depicts a specific quantity of each component when 1000 g in total of the cosmetic composition according to Embodiment 4 mentioned above is formed. Example 4-3 in FIG. 15 depicts a specific quantity of each component when 1500 g in total of the cosmetic composition according to Embodiment 4 mentioned above is formed. Example 4-4 in FIG. 15 depicts a specific quantity of each component when 2000 g in total of the cosmetic composition according to Embodiment 4 mentioned above is formed.

The specific amounts of the components in the cosmetic composition of the present invention will become obvious with reference to Examples 4-1 to 4-4 illustrated in FIG. 14. The cosmetic composition according to each example can also exert the working effects described for the embodiment mentioned above, irrespective of the number of grams of the total quantity, as long as the cosmetic composition has the component ratios shown in FIG. 13, of Embodiment 4 mentioned above.

The cosmetic of the present invention can be formed by further adding an additional component to the cosmetic composition having the component ratios according to the formulation of the invention shown in Embodiment 4 mentioned above.

### [Cosmetic of Embodiment 4]

Specifically, the cosmetic of Embodiment 4 having the efficacy of the cosmetic composition according to Embodiment 4 can be formed by adding one or more members selected from histidine, proline, isoleucine, serine, valine, aspartic acid, threonine, phenylalanine, PCA, PCA-Na, Na lactate, and water to the cosmetic composition of Embodiment 4 mentioned above. FIG. 15 shows the component ratios of the cosmetic of Embodiment 4 which is configured so as to comprise the cosmetic composition according to Embodiment 4. FIG. 15 is a diagram showing the component ratios of the cosmetic according to Embodiment 4 that can be formed by adding predetermined component(s) to the cosmetic composition according to Embodiment 4 shown in FIG. 13.

As shown in FIG. 15, the component ratios of the cosmetic according to Embodiment 4 are such that
the amount of the glycine is 25% by mass based on the total mass of the cosmetic,
the amount of the Na citrate is 19.2% by mass based on the total mass of the cosmetic,
the amount of the arginine is 16.7% by mass based on the total mass of the cosmetic,
the amount of the alanine is 13.4% by mass based on the total mass of the cosmetic,
the amount of the 2Na succinate is 13.4% by mass based on the total mass of the cosmetic, and
the amount of the tartaric acid is 12.18% by mass based on the total mass of the cosmetic, and
0.01% by mass each of all of histidine, proline, isoleucine, serine, valine, aspartic acid, threonine, phenylalanine, PCA, PCA-Na, Na lactate, and water is further contained based on the total mass of the cosmetic. The cosmetic of Embodiment 4 can be configured by these component ratios and therefore produced as a cosmetic having preferred efficacy.

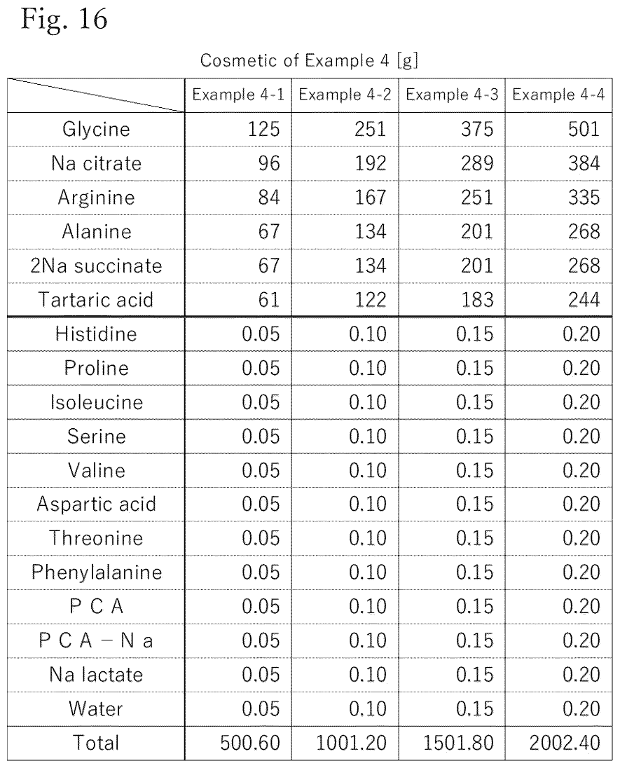

Next, specific examples of the cosmetic of Embodiment 4 will be given with reference to FIG. 16. FIG. 16 is a diagram showing Examples 4-1 to 4-4 which are specific examples of the cosmetic according to Embodiment 4 shown in FIG. 15.

### [Examples of Cosmetic of Embodiment 4]

FIG. 16 illustrates specific amounts of the components for forming a cosmetic having component ratios according to the inventive formulation shown in Embodiment 4 mentioned above. For example, FIG. 16 indicates a specific quantity by the number of grams as Example 4-1, of a cosmetic formed by forming 500 g of the cosmetic composition according to Embodiment 4 shown in FIG. 14 and adding all of histidine, proline, isoleucine, serine, valine, aspartic acid, threonine, phenylalanine, PCA, PCA-Na, Na lactate, and water to the cosmetic composition so that the component ratios in FIG. 15 are met. Likewise, Example 4-2 in FIG. 16 depicts a specific quantity by a specific number of grams, of a cosmetic formed by forming 1000 g of the cosmetic composition according to Embodiment 4 mentioned above and adding all of histidine, proline, isoleucine, serine, valine, aspartic acid, threonine, phenylalanine, PCA, PCA-Na, Na lactate, and water to the cosmetic composition so that the component ratios in FIG. 15 are met. Likewise, Example 4-3 in FIG. 16 depicts a specific quantity by a specific number of grams, of a cosmetic formed by forming 1500 g of the cosmetic composition according to Embodiment 4 mentioned above and adding all of histidine, proline, isoleucine, serine, valine, aspartic acid, threonine, phenylalanine, PCA, PCA-Na, Na lactate, and water to the cosmetic composition so that the component ratios in FIG. 15 are met. Likewise, Example 4-4 in FIG. 16 depicts a specific quantity by a specific number of grams, of a cosmetic formed by forming 2000 g of the cosmetic composition according to Embodiment 4 mentioned above and adding all of histidine, proline, isoleucine, serine, valine, aspartic acid, threonine, phenylalanine, PCA, PCA-Na, Na lactate, and water to the cosmetic composition so that the component ratios in FIG. 15 are met.

The specific amounts of the components in the cosmetic of Embodiment 4 will become obvious with reference to Examples 4-1 to 4-4 illustrated in FIG. 16. The cosmetic according to each example can also exert the working effects described for the embodiment mentioned above, irrespective of the number of grams of the total quantity, as long as the cosmetic has the component ratios shown in FIG. 15, of Embodiment 4 mentioned above.

The cosmetic composition according to Embodiment 4 and the cosmetic using this cosmetic composition are described with reference to FIG. 13 to FIG. 16.

The preferred embodiments of the present invention are described above with reference to FIG. 1 to FIG. 16. However, the technical scope of the present invention is not limited to the scope described in each of the embodiments described above. Various changes or modifications can be made in each of the embodiments described above. It is obvious from the description of the scope of claims that modes with such changes or modifications may be included in the technical scope of the present invention.

The cosmetic composition of the present invention exerts efficacy when applied to hair, the skin, hair of the head and/or the scalp. Use of the method for applying a cosmetic composition or a cosmetic of the present invention for hair, the skin, hair of the head and/or the scalp is useful in fields, for example, hair dressing, beauty, and cosmetics.

What is claimed is:

1. A cosmetic composition comprising:
tartaric acid;
glycine;
Na citrate;
alanine;
2Na succinate; and
Na sulfate, wherein
an amount of the tartaric acid is 57.07% by mass based on a total mass of the composition,
an amount of the glycine is 13.42% by mass based on the total mass of the composition,
an amount of the Na citrate is 10.41% by mass based on the total mass of the composition,
an amount of the alanine is 7.61% by mass based on the total mass of the composition,
an amount of the 2Na succinate is 7.48% by mass based on the total mass of the composition, and
an amount of the Na sulfate is 4.01% by mass based on the total mass of the composition.

2. The cosmetic according to claim 1, further comprising at least one component selected from the group consisting of histidine, proline, isoleucine, serine, valine, aspartic acid, threonine, phenylalanine, arginine, PCA, PCA-Na, Na lactate, and water.

3. The cosmetic according to claim 2, wherein
an amount of the tartaric acid is 57% by mass based on the total mass of the cosmetic,
an amount of the glycine is 13.4% by mass based on the total mass of the cosmetic,
an amount of the Na citrate is 10.4% by mass based on the total mass of the cosmetic,
an amount of the alanine is 7.6% by mass based on the total mass of the cosmetic,
an amount of the 2Na succinate is 7.47% by mass based on the total mass of the cosmetic, and
an amount of the Na sulfate is 4% by mass based on the total mass of the cosmetic.

4. The cosmetic according to claim 3, wherein
the at least one component includes all of the histidine, the proline, the isoleucine, the serine, the valine, the aspartic acid, the threonine, the phenylalanine, the arginine, the PCA, the PCA-Na, the Na lactate, and the water, and
an amount of each of the histidine, the proline, the isoleucine, the serine, the valine, the aspartic acid, the threonine, the phenylalanine, the arginine, the PCA, the PCA-Na, the Na lactate, and the water is 0.01% by mass based on the total mass of the cosmetic.

5. A cosmetic composition comprising:
arginine;
Na citrate;
glycine;
alanine;
trehalose, and
2Na succinate, wherein
an amount of the arginine is 72.46% by mass based on a total mass of the composition,
an amount of the Na citrate is 8.30% by mass based on the total mass of the composition,
an amount of the glycine is 4.81% by mass based on the total mass of the composition,
an amount of the alanine is 4.81% by mass based on the total mass of the composition,
an amount of the trehalose is 4.81% by mass based on the total mass of the composition, and
an amount of the 2Na succinate is 4.81% by mass based on the total mass of the composition.

6. The cosmetic according to claim 5, further comprising at least one component selected from the group consisting of histidine, proline, isoleucine, serine, valine, aspartic acid, threonine, phenylalanine, PCA, PCA-Na, Na lactate, and water.

7. The cosmetic according to claim 6, wherein
an amount of the arginine is 72.38% by mass based on the total mass of the cosmetic,
an amount of the Na citrate is 8.3% by mass based on the total mass of the cosmetic,
an amount of the glycine is 4.8% by mass based on the total mass of the cosmetic,
an amount of the alanine is 4.8% by mass based on the total mass of the cosmetic,
an amount of the trehalose is 4.8% by mass based on the total mass of the cosmetic, and
an amount of the 2Na succinate is 4.8% by mass based on the total mass of the cosmetic.

8. The cosmetic according to claim 7, wherein
the at least one component includes all of the histidine, the proline, the isoleucine, the serine, the valine, the aspartic acid, the threonine, the phenylalanine, the PCA, the PCA-Na, the Na lactate, and the water, and
an amount of each of the histidine, the proline, the isoleucine, the serine, the valine, the aspartic acid, the threonine, the phenylalanine, the PCA, the PCA-Na, the Na lactate, and the water is 0.01% by mass based on the total mass of the cosmetic.

9. A cosmetic composition comprising:
tartaric acid;
glycine;
Na citrate;
alanine;
2Na succinate;
arginine, and
Na sulfate, wherein
an amount of the tartaric acid is 28.04% by mass based on a total mass of the composition,
an amount of the glycine is 21.73% by mass based on the total mass of the composition,
an amount of the Na citrate is 17.02% by mass based on the total mass of the composition,
an amount of the alanine is 12.01% by mass based on the total mass of the composition,
an amount of the 2Na succinate is 12.01% by mass based on the total mass of the composition,
an amount of the arginine is 5.19% by mass based on the total mass of the composition, and
an amount of the Na sulfate is 4.00% by mass based on the total mass of the composition.

10. The cosmetic according to claim 9, further comprising at least one component selected from the group consisting of histidine, proline, isoleucine, serine, valine, aspartic acid, threonine, phenylalanine, PCA, PCA-Na, Na lactate, and water.

11. The cosmetic according to claim 10, wherein
an amount of the tartaric acid is 28% by mass based on the total mass of the cosmetic,
an amount of the glycine is 21.7% by mass based on the total mass of the cosmetic,
an amount of the Na citrate is 17% by mass based on the total mass of the cosmetic,
an amount of the alanine is 12% by mass based on the total mass of the cosmetic,
an amount of the 2Na succinate is 12% by mass based on the total mass of the cosmetic,
an amount of the arginine is 5.18% by mass based on the total mass of the cosmetic, and
an amount of the Na sulfate is 4% by mass based on the total mass of the cosmetic.

12. The cosmetic according to claim 11, wherein
the at least one component includes all of the histidine, the proline, the isoleucine, the serine, the valine, the aspartic acid, the threonine, the phenylalanine, the PCA, the PCA-Na, the Na lactate, and the water, and
an amount of each of all of the histidine, the proline, the isoleucine, the serine, the valine, the aspartic acid, the threonine, the phenylalanine, the PCA, the PCA-Na, the Na lactate, and the water is 0.01% by mass based on the total mass of the cosmetic.

13. A cosmetic composition comprising:
glycine;
Na citrate;
arginine;
alanine;
2Na succinate; and
tartaric acid, wherein
an amount of the glycine is 25.03% by mass based on a total mass of the composition,
an amount of the Na citrate is 19.22% by mass based on the total mass of the composition,
an amount of the arginine is 16.72% by mass based on the total mass of the composition,
an amount of the alanine is 13.42% by mass based on the total mass of the composition,
an amount of the 2Na succinate is 13.42% by mass based on the total mass of the composition, and
an amount of the tartaric acid is 12.19% by mass based on the total mass of the composition.

14. The cosmetic according to claim 13, further comprising at least one component selected from the group consisting of histidine, proline, isoleucine, serine, valine, aspartic acid, threonine, phenylalanine, PCA, PCA-Na, Na lactate, and water.

15. The cosmetic according to claim 14, wherein
an amount of the glycine is 25% by mass based on the total mass of the cosmetic,
an amount of the Na citrate is 19.2% by mass based on the total mass of the cosmetic,
an amount of the arginine is 16.7% by mass based on the total mass of the cosmetic,
an amount of the alanine is 13.4% by mass based on the total mass of the cosmetic,
an amount of the 2Na succinate is 13.4% by mass based on the total mass of the cosmetic, and
an amount of the tartaric acid is 12.18% by mass based on the total mass of the cosmetic.

16. The cosmetic according to claim 15, wherein
the at least one component includes all of the histidine, the proline, the isoleucine, the serine, the valine, the aspartic acid, the threonine, the phenylalanine, the PCA, the PCA-Na, the Na lactate, and the water, and
an amount of each of the histidine, the proline, the isoleucine, the serine, the valine, the aspartic acid, the threonine, the phenylalanine, the PCA, the PCA-Na, the Na lactate, and the water is 0.01% by mass based on the total mass of the cosmetic.

17. A method for applying a cosmetic composition to hair, skin, hair of a head and/or a scalp, the method comprising applying the cosmetic composition according to claim 1 to the hair, the skin, the hair of the head and/or the scalp.

18. A method for applying a cosmetic to hair, skin, hair of a head and/or a scalp, the method comprising applying the cosmetic according to claim 1 to the hair, the skin, the hair of the head and/or the scalp.
